(19) United States
(12) Patent Application Publication
(10) Pub. No.: US 2022/0241725 A1
(43) Pub. Date: Aug. 4, 2022

(54) METHOD AND APPARATUS FOR ALKANE OXIDATION

(71) Applicant: The Queen's University of Belfast, Belfast, Antrim (GB)

(72) Inventors: David Rooney, Belfast, Antrim (GB); Kevin Morgan, Belfast, Antrim (GB)

(21) Appl. No.: 17/432,251

(22) PCT Filed: Feb. 24, 2020
(86) PCT No.: PCT/EP2020/054794

§ 371 Date: Aug. 19, 2021

(30) Foreign Application Priority Data
Feb. 25, 2019 (GB) 1902506.3

(51) Int. Cl. B01D 53/86; B01D 53/88; B01D 53/94; B01D 53/00; B01J 29/44; B01J 21/06; B01J 21/04; B01J 35/04; B01J 35/00; F01N 3/28; F01N 3/20

(52) U.S. Cl. CPC B01D 53/864; F01N 2370/04; B01D 53/944; B01D 53/007; B01J 29/44; B01J 21/063; B01J 21/04; B01J 35/04; B01J 35/004; F01N 3/2803; F01N 3/2086; B01D 2255/504; B01D 2255/1023; B01D 2255/1021; B01D 2255/20707; B01D 2255/802; B01D 2257/7025; B01D 2259/804; B01D 53/885

(57) ABSTRACT

The present invention provides a method for catalytic oxidation of alkanes, where the catalyst comprises a photoactive material that is activated when the catalyst is irradiated with UV light. In particular, the method is for the catalytic oxidation of a C1-C5 alkane using an oxidation catalyst comprising a photoactive material, said method comprising the steps of a) activating the photoactive material by irradiating the catalyst with UV light and b) contacting the activated catalyst with a gaseous feedstream comprising an amount of C1-C5 alkane at a temperature of from 150° C. to 600° C.

The present invention relates to oxidation of alkanes. In particular, the present invention relates to catalytic oxidation of alkanes, where the catalyst comprises a photoactive material that is activated when the catalyst is irradiated with UV light.

Large amounts of air pollution are believed to result from the use of hydrocarbon fuels in internal combustion engines, and in particular diesel engines. Emissions from such engines are known to significantly contribute to air pollution, for example in the form of greenhouse gases or by the formation of smog.

Dual-Fuel Technology, which can substitute up to 90% of diesel with natural gas, or dedicated natural gas engines, offer the potential to reduce emissions (up to 25% less carbon per kWh), with the additional economic benefit of natural gas costing much less per litre equivalent of diesel. In addition, when using primarily methane-based fuels such as natural gas and/or bio-methane, there are typically significantly less particulates formed than there are using petrol, diesel or other bio-fuels.

Nonetheless, due to strict emissions regulations, emission of hydrocarbons from engines, and methane in particular, pose a problem for the widespread use of such fuels. Despite natural gas vehicles offering potential reductions in some emissions (e.g. CO₂ and particulate matter), methane is 25-30 times worse for the environment as a greenhouse gas compared to CO₂. Typically, even the best combustion engines only achieve 98% combustion efficiency of natural gas. In some cases this is not sufficient, even with existing catalysts, to reduce emissions and meet legislative limits.

Undesired hydrocarbon emissions can also be produced when using conventional petrol and diesel engines, and there is a particular global focus on reducing diesel emissions following the 2017 diesel emissions scandal.

Accordingly, there is therefore a need for ways to provide stable and high efficiency removal of unburned hydrocarbons such as methane, for example for incorporation into the exhaust of engines.

The principal challenges for methane oxidation are the temperature required and catalyst deactivation. It is believed that the main reason for deactivation of typical methane oxidation catalysts is the presence of water vapour, for example in exhaust streams. This results in significant decreases in the conversion of methane in relatively short periods of time, meaning that, in the case of exhaust emissions from engines, legislative limits are typically exceeded well within the lifetime of the engine. While it is possible to regenerate these catalysts to an extent, full activity is never recovered and the deactivation will always reoccur, with any recovered activity typically lower than prior to the regeneration.

In addition, methane oxidation typically requires high temperatures of at least 400° C., which can reduce the effectiveness of pollutant reducing systems under conditions where feed streams, such as exhaust gases, are supplied at lower temperatures. Therefore, there is also a need for ways to more effectively remove hydrocarbons from feed streams that are provided at temperatures typical of exhaust streams from hydrocarbon combustion processes.

It has been surprisingly found that problems of deactivation associated with the presence of water in the feedstream of a hydrocarbon oxidation process can be addressed by using an oxidation catalyst comprising a photoactive material, which may be activated with UV (ultraviolet) light, and contacting the feedstream with the catalyst in a particular temperature range. It has also been found that the oxidation of hydrocarbons may be advantageously conducted at temperatures typically associated with hydrocarbon combustion exhaust streams.

Thus, according to a first aspect of the invention, there is provided a method for the catalytic oxidation of a C1-C5 alkane using an oxidation catalyst comprising a photoactive material, said method comprising the steps of:
a) activating the photoactive material by irradiating the catalyst with UV light; and
b) contacting the activated catalyst with a gaseous feedstream comprising an amount of C1-C5 alkane at a temperature of from 150° C. to 600° C.

In a further aspect, the present invention provides an apparatus for use in catalytic oxidation of C1-C5 alkane present in a gaseous feedstream, said apparatus comprising:

a) an oxidation catalyst configured for catalytic oxidation of C1-C5 alkane present in a gaseous feedstream at temperatures of up to 600° C., wherein the oxidation catalyst comprises a photoactive material;

b) a UV light generating means configured for irradiation of the photoactive material; and
c) a housing within which the oxidation catalyst is disposed and within which UV light from the UV light generating means may be transmitted, which housing is configured to receive a supply of the gaseous feedstream comprising C1-C5 alkane.

In another aspect, the present invention provides an exhaust system for an internal combustion engine for powering an automotive vehicle, locomotive vehicle or marine vessel comprising an apparatus described herein

By using an oxidation catalyst comprising a photoactive material, activating the photoactive material using UV light and contacting the catalyst with the feedstream at 150° C. to 600° C. in accordance with the method of the invention, the stability of the catalyst in the presence of water and/or at higher temperatures may be improved. Without wishing to be bound by any particular theory, it is believed that by activating the photoactive material with UV light, water adsorption onto active oxidation catalyst sites can be at least partially prevented by reduction of water on the photoactive material to produce hydrogen. The production of hydrogen by this mechanism may also be beneficial as hydrogen is known to promote the oxidation of hydrocarbons. Again, without wishing to be bound by any particular theory, it is believed that hydrogen may promote hydrocarbon oxidation either by exothermic reaction to increase local temperature, or by promoting reduction of metal oxides in the catalyst to more active species.

The photoactive material referred to herein will be understood to refer to a photocatalytic material that shows activity under UV irradiation, i.e. a material that is activated, at least in part, by irradiation with UV light. Without wishing to be bound by any particular theory, it will be appreciated that photoactive materials are typically activated by excitation of electrons to form positively charged “holes” in the valence band of the material. Reactions may then take place between the excited electrons or the holes and chemical species to form radicals.

Oxidation of a C1-C5 alkane as referred to herein will be understood to relate primarily to the complete oxidation of the alkane to produce CO₂ and water, and/or incomplete oxidation of the alkane to produce CO and hydrogen.

In some preferred embodiments, activation of the photoactive material in part (a) may be performed in the absence of the gaseous feedstream comprising an amount of C1-C5 alkane. For example, the activation may be performed in the presence of air or under at least partial vacuum. In other preferred embodiments, the photoactive material may be activated in step (a) in the presence of the gaseous feedstream comprising an amount of C1-C5 alkane.

The photoactive material is preferably activated in step a) in the substantial absence of water vapour, for example in the presence of a stream containing less than 5% by volume of water vapour, preferably less than 2% by volume water vapour, more preferably less than 1% by volume water vapour. In some preferred embodiments, the photoactive material is activated in step (a) in the presence of a substantially dry feed, for example a feed containing less than 0.5% water vapour and preferably less than 0.1% water vapour, or under reduced pressure. It will be appreciated that the water content of a stream may be determined in any suitable way, and such methods are known to the person of skill in the art. For example, water content may be measured directly by infra-red or mass spectrometry, or may be indirectly measured by mass balance calculations. Water content may also be measured using a hygrometer, and such hygrometers are known in the art.

In some embodiments, the activation in step (a) is conducted under a static atmosphere, which may be substantially dry as defined previously. For example, where the feed stream comprises exhaust gases from an engine, the activation may be conducted at least in part before gases from the engine reach the catalyst, for example prior to or as part of the start-up process of the engine.

Preferably, the photoactive material of the oxidation catalyst is irradiated intermittently or continuously with UV light during contact with the gaseous feedstream comprising an amount of C1-C5 alkane in step b). It will be appreciated that intermittently irradiating the photoactive material may permit continuous activity of the photoactive material to be maintained, whilst saving energy by avoiding constant irradiation. It will be appreciated that the timing of the intermittent irradiation will depend on the lifetime of the oxidation catalyst, and this may vary depending on the particular catalyst used. In some instances, the photoactive material may initially be irradiated in step a) for a period of at least about 10 minutes, preferably at least about 30 minutes, and up to about 2 hours, preferably up to about 1 hour, followed by operation of step b) without further irradiation for at least about 3 hours, preferably at least about 4 hours. It will also be appreciated that any intermittent irradiation steps following an initial activation may be substantially the same as described herein in relation to the irradiation used in step a), and for such further irradiation steps, the conditions will typically be the same as those applied in step b).

The UV light used in the method may be any suitable frequency, and it will be understood that UV light as referred to herein will mean light having a wavelength in the range of from 10 nm to 450 nm. In preferred embodiments, the irradiation in step a) is with UV radiation having a wavelength of from 150 to 450 nm, preferably from 280 to 450 nm, more preferably from 350 to 400 nm, for example from 375 to 395 nm.

The UV light may be provided by any suitable UV generating means, for example fluorescent UV lamps, incandescent lamps such as halogen lamps, gas-discharge lamps and LEDs. Preferably the means for generating UV light comprises an LED. Any suitable LED UV light source may be used and such LED UV sources are known to one of skill in the art. Suitable LED sources may include, for example, semiconductor p-n junction devices that may comprise materials such as gallium arsenide (GaAs), gallium arsenide phosphide (GaAsP), gallium phosphide (GaP), or indium gallium nitride (InGaN). In some embodiments, the means for generating UV light is a laser. As will be appreciated, the UV generating means will be suitable for generating light having wavelengths as referred to previously herein.

It will be understood that the means for generating the UV light may be configured so as to directly or indirectly irradiate the photoactive material and thus the means for generating the UV light may be provided at the location of the photoactive material (i.e. in situ) or at a location remote from the photoactive material. It also will be appreciated that the UV light irradiated onto the photoactive material may be provided from multiple UV light generating means, for example in the form of an array of UV sources.

As will also be appreciated, where the UV generating means is remote from the photoactive material, the UV light may be transmitted from the generating means to the photoactive material using optics, for example by optical fibre. In preferred embodiments, the UV light is transmitted to the photoactive material by one or more optical fibres. By using optical fibres to deliver the UV light remotely, any disruption of feed gas flow over the catalyst that could be caused by the generating means being located in situ may be avoided, and the UV light may be more easily delivered to inaccessible areas of a catalyst surface, for example to multiple positions inside a catalyst monolith. In addition, this allows the UV generating means and any electronics to avoid exposure to the heat associated with the catalyst so as to reduce heat-related damage or degradation of the UV generating means and to ensure its reliable operation.

The concentration of C1-C5 alkanes in the feedstream is not particularly limited and it will be appreciated that the concentration may vary depending on the particular source of the gaseous feedstream. In preferred embodiments, the gaseous feedstream comprises from 0.01 to 20% by volume of C1-C5 alkane, preferably from 0.1 to 10.0% by volume, more preferably from 0.5 to 5.0% by volume of C1-C5 alkane.

A C1-C5 alkane as referred to herein will be understood to mean a hydrocarbon having the formula CₙH₂ₙ₊₂, where n is from 1 to 5. Preferably, the C1-C5 alkane is selected from C1-C3 alkanes and combinations thereof, more preferably the C1-C5 alkane is selected from methane, propane or a combination thereof, even more preferably the C1-C5 alkane is methane.

Thus in preferred embodiments, the gaseous feedstream comprises at least 0.01% by volume of methane, preferably at least 0.2% by volume of methane, more preferably at least 0.3% by volume of methane, for example at least 0.5% by volume of methane.

It will be appreciated that, in comparison to other hydrocarbons, methane is stable and more difficult to oxidise. For example, higher temperatures of at least 400° C. are typically required for methane oxidation. Using the present method, it has surprisingly been found that oxidation of methane may be conveniently conducted at lower temperatures.

The feedstream may contain water vapour, and it will be appreciated that feedstreams comprising exhaust gases will typically contain an amount of water vapour. In preferred embodiments, the feedstream comprises from 4.0% to 20.0% by volume of water vapour, preferably from 5.0 to 15.0% by volume of water vapour, more preferably from 5.0% to 10.0% by volume of water vapour. As described previously, using the present method may advantageously reduce or avoid problems of deactivation associated with the presence of water in the feedstream of a hydrocarbon oxidation process.

While the methods described herein may be particularly advantageous for the treatment of water containing feedstreams, it will be appreciated that in some embodiments the amount of water may be limited. Accordingly, in some preferred embodiments, the feedstream may contain less than 5% by volume of water vapour, for example less than 3% by volume or less than 1% by volume.

In general, it will be appreciated that the overall composition of the feedstream may vary depending on the source of the feed. Typically, in addition to the possible components described previously herein, the feedstream may comprise gases found in air and/or those produced by combustion processes. For example, the feedstream may comprise gases such as nitrogen, oxygen, carbon dioxide and in some instances carbon monoxide. In particular, the feedstream may comprise oxygen in an amount up to 15% by volume, and preferably from 1 to 15% by volume. The feedstream may also comprise carbon dioxide in an amount of from 1 to 20% by volume, for example from 10 to 15% by volume. It will be appreciated that the balance of the feed will typically be made up of nitrogen. In particular, the feedstream may comprise about 20 to 80% by volume of nitrogen, for example from 50% to 80% by volume of nitrogen, preferably from 70 to 80% by volume of nitrogen.

Where the feed comprises an exhaust stream from a combustion engine, it will be appreciated that the composition of the feed may generally depend on the air to fuel ratio in the engine. Preferably, where the feed comprises an exhaust stream from an internal combustion engine, the air to fuel ratio gives a λ (lambda) value (air-fuel equivalence ratio) of from about 1 to about 4. It will also be appreciated that the composition of a feed comprising an exhaust stream from an internal combustion engine may vary depending on the combustion efficiency of the engine, which for non-stationary engines, for example in vehicles, can typically vary during use depending on how the engine is being operated.

The contacting step b) is suitably conducted at a temperature of 150° C. to 600° C. Preferably, contacting step b) is conducted at a temperature of at least 175° C., preferably at least 200° C., more preferably at least 225° C., even more preferably at least 250° C. In some instances the temperature may be at least 270° C.

It will be appreciated that the temperature at which contacting step b) is conducted may vary depending on the nature of the feed, for example the source of the feed or the composition of the feed. In some preferred embodiments, where the feedstream comprises water vapour the contacting step b) is conducted at a temperature of at least 200° C., for example, where the feedstream comprises greater than 5% by volume water vapour, the contacting step b) may be conducted at a temperature of at least 250° C., for example at least 270° C.

It will be appreciated that the temperature at which activating step a) is conducted may be any suitable temperature, and where the feed stream is an exhaust stream from an internal combustion engine, the temperature may be the temperature of exhaust gas during a cold start of the engine, for example from about 10 to about 60° C. In some embodiments, activating step a) may be conducted at ambient temperature (typically from about 10 to about 30° C.). In some embodiments, the temperature at which activating step a) is conducted may be as described in relation to contacting step b). For example, the activating step a) may be conducted at the same temperature as is applied in step b), or may be conducted whilst heating from a lower temperature to the temperature at which step b) is conducted. For instance, where the feed stream is an exhaust stream from an internal combustion engine, the activating step a) as well as step b) may be conducted at exhaust temperatures. Where an additional re-activation step is employed, for instance as one or more intermittent irradiation steps applied whilst carrying out step b), it will be appreciated that the temperature will be the same as is used in step b).

Steps a) and b) may suitably be conducted at any pressure, for example at around atmospheric pressure, i.e. around 1 bar absolute. Preferably the pressure is less than 5 bar absolute, more preferably less than 2 bar absolute, for example from 1 to 1.5 bar absolute. In some embodiments, step (a) may be conducted at reduced pressure, i.e. less than 1 bar absolute.

Contacting step b) may be conducted as a continuous process by continuously passing the feedstream over the oxidation catalyst. The flow rate of the feedstream over the catalyst may be any suitable flow, and it will be appreciated that the flow rate may vary depending on the source of the feed. The flow rate of the gaseous feedstream in terms of Gas Hourly Space Velocity (GHSV) (volume of gaseous feed stream/total volume of catalyst/hour) is suitably in the range of from 50 to 60,000 h⁻¹, for example from 20,000 to 50,000 h⁻¹.

The benefits of the present invention are believed to derive at least in part from the hitherto unknown combination of thermal alkane oxidation under the influence of both oxidative catalysis at elevated temperature together with photocatalysis provided by a photoactive material. The oxidation catalyst comprising a photoactive material may be any suitable catalyst that may be used for catalysing alkane oxidation at temperatures in accordance with the method of the invention.

It will be appreciated that the oxidation catalyst typically comprises a thermally active component that is capable of the oxidation of alkanes at high temperatures, in addition to the photoactive material. Preferably, the oxidation catalyst comprises one or more metals selected from ruthenium, palladium, platinum, gold, silver, rhodium, iridium, rhenium, manganese, chromium, nickel, iron, molybdenum, tungsten, zirconium, gallium, thorium, lanthanum, cerium and mixtures thereof. More preferably, the oxidation catalyst comprises palladium. In other preferred embodiments, the oxidation catalyst comprises at least two different metals, for example, the oxidation catalyst may comprise an additional metal as, for example a promoter, preferably the two metals are selected from palladium, platinum, gold, silver, rhodium, iridium and rhenium, more preferably the at least two different metals includes palladium and platinum. These combinations of metals have been found to be particularly effective at increasing alkane conversion efficiency at the lower end of the contacting temperatures that may be used in accordance with step b) of the method of the invention.

Preferably the photoactive material comprises a photoactive material selected from transition metal oxides, and combinations thereof. In particularly preferred embodiments, the photocatalyst comprises a photoactive material selected from TiO₂, WO₂, CoO and combinations thereof. Most preferably, the photoactive material comprises, or consists essentially of, TiO₂. The photoactive material may be formed in situ from a precursor, for example a colloidal mixture comprising anatase and rutile.

Suitably, the oxidation catalyst may be unsupported or may be supported on a support material. The photoactive material may suitably be incorporated into the oxidation catalyst to form a composite in the sense that atoms of a metal of the oxidation catalyst replace atoms in the structure of the photoactive material or a support material, or alternatively the metal may be incorporated in other ways, for example by surface deposition on the photoactive material or support material.

In preferred embodiments, the support material is selected from silica (e.g. fumed silica), alumina, aluminosilicate such as zeolites (e.g. ZSM-5 or mordenite), silica-alumina, ceria, titania, gallia, zirconia, magnesia, yttria, zinc oxide, activated carbon, silicon carbide, titanium carbide, fluoropolymer resins such as Nafion NR50, and mixtures thereof. In preferred embodiments, the support material is alumina, aluminosilicate such as zeolite or mordenite, zirconia, ceria, silica (e.g. fumed silica), fluoropolymer resins such as Nafion NR50, and combinations thereof. Preferably, the support material is alumina or zeolite such as ZSM-5.

The support material may suitably be in the form of a powder, granulate, pellet, extrudate, or combinations thereof. In some embodiments, the unsupported photocatalyst may be formed as a powder, granulate, pellet, extrudate, or combinations thereof.

The supported oxidation catalyst may be prepared by any suitable method known to the person skilled. For example, it may be prepared by impregnation, precipitation or gelation. The oxidation catalyst may also be prepared by mulling or kneading a support material with either soluble or insoluble precursor compounds of the oxidation catalyst, before extruding, drying and calcining the product. Preferably, the oxidation catalyst is prepared using a wet impregnation method, which may be aided by sonication. It will be appreciated that precursor compounds of the oxidation catalyst mentioned herein refers to one or more precursor compounds that can form the photoactive material as well as the thermally active component.

A suitable impregnation method, for example, comprises impregnating a support material with the precursor compound of the photoactive material and/or thermally active component which is thermally decomposable to the oxide and/or metallic form. It will be appreciated that the photoactive material and/or thermally active component may also preferably be impregnated onto the support in its active form, for example as a metal oxide. Preferably, the photoactive material is impregnated onto the support in the form of a metal oxide. Any suitable impregnation technique including the incipient wetness technique or the excess solution technique, both of which are well-known in the art, may be employed. The incipient wetness technique is so-called because it requires that the volume of impregnating solution be predetermined so as to provide the minimum volume of solution necessary to just wet the entire surface of the support, with no excess liquid. The excess solution technique as the name implies, requires an excess of the impregnating solution, the solvent being thereafter removed, usually by evaporation.

The impregnation solution or suspension may suitably be either an aqueous or a non-aqueous, organic solution or suspension of the precursor compound or the photoactive material. Suitable non-aqueous organic solvents include, for example, alcohols, ketones, liquid paraffinic hydrocarbons and ethers. Alternatively, aqueous organic solutions or suspensions, for example an aqueous alcoholic solution or suspension may be employed.

Impregnation may be conducted with a support material which is in a powder, granular or pelletized form. Alternatively, impregnation may be conducted with a support material which is in the form of a shaped extrudate. The aforementioned impregnation of a shaped extrudate refers to chemically supporting the catalyst on the surface of an extrudate, for example an extrudate comprising one or more shaped articles of a support material as described previously, for example shaped articles of alumina or zeolite.

Alternatively, where a preformed physical support, which may in some instances be an extrudate, is coated (i.e. physically rather than chemically supporting), it will be appreciated that the physical support may be contacted with a coating solution or suspension comprising the oxidation catalyst, or precursors thereof, by any suitable means including, for instance, by wash coating. Example methods of coating a monolith physical support are also described in particular below.

Where a powder or granulate of support material is impregnated, the powder or granulate may be admixed with the impregnating solution or suspension by any suitable means of which the skilled person is aware, such as by adding the powder or granulate to a container of the impregnating solution or suspension and stirring or sonicating.

Where an extrusion step immediately follows impregnation of a powder or granulate, the mixture of powder or granulate and impregnating solution or suspension may be further processed if it is not already in a form which is suitable for extruding. For instance, the mixture may be mulled to reduce the presence of larger particles that may not be readily extruded, or the presence of which would otherwise compromise the physical properties of the resulting extrudate. Mulling typically involves forming a paste which is suitable for shaping by extrusion. Any suitable mulling or kneading apparatus or method of which the skilled person is aware may be used for mulling in the context of the present invention. For example, a pestle and mortar may suitably be used in some applications or a mechanical or powered muller may suitably be employed. It will be appreciated that complete removal of bound solvent from the impregnation solution or suspension may be conducted to effect complete precipitation after extrusion.

In embodiments where a calcination step is performed on an impregnated powder or granulate, thereby completely removing solvent of the impregnation solution or suspension, the calcined powder or granulate may also be further processed in order to form a mixture which is suitable for extruding. For instance, an extrudable paste may be formed by combining the calcined powder or granulate with a suitable solvent, for example a solvent used for impregnation, and mulled as described above.

In some embodiments, an extrudate or other preformed chemical support that has been impregnated with catalyst is converted into a powder or granulate. This may be achieved by any suitable means of which the person of skill in the art is aware. For instance, the impregnated support material, which may in some embodiments be a dry extrudate, may be crushed and/or ground/milled.

Preferred support materials are substantially free of extraneous metals or elements which might adversely affect the catalytic activity of the system. Thus, preferred support materials are at least 95% w/w pure, more preferably at least 99% w/w pure. Impurities preferably amount to less than 1% w/w, more preferably less than 0.60% w/w and most preferably less than 0.30% w/w. The pore volume of the support is preferably more than 0.10 ml/g and preferably more than 0.15 ml/g. The average pore radius (prior to impregnation with the photoactive material or dopant metal) of the support material is generally from 10 to 500 Å, preferably from 15 to 100 Å, more preferably from 20 to 80 Å and most preferably from 25 to 40 Å. The BET surface area is suitably from 2 to 1000 m²g, preferably from 10 to 600 m²/g, more preferably from 300 to 600 m²/g, and most preferably 350 to 500 m²/g.

The BET surface area, pore volume, pore size distribution and average pore radius may be determined from the nitrogen adsorption isotherm determined at 77K using, for example, a Micromeritics TRISTAR 3000 static volumetric adsorption analyser. A procedure which may be used is an application of British Standard methods BS4359:Part 1:1984 ‘Recommendations for gas adsorption (BET) methods’ and BS7591:Part 2:1992, ‘Porosity and pore size distribution of materials’—Method of evaluation by gas adsorption. The resulting data may be reduced using the BET method (over the pressure range 0.05-0.20 P/Po) and the Barrett, Joyner & Halenda (BJH) method (for pore diameters of 20-1000 Å) to yield the surface area and pore size distribution respectively. Suitable references for the above data reduction methods are S. Brunauer, P.H. Emmett & E. Teller, J. Amer. Chem. Soc. 60, p 309 (1938) and E. P. Barrett, L. G. Joyner, P. P. Halenda, J. Am Chem. Soc., 73, p 373 (1951).

In preferred embodiments, where a powder of support material is employed, the powder has a median particle size diameter (d50) of less than 50 μm, preferably less than 25 μm. Particle size diameter (d50) may suitably be determined by means of a particle size analyser (e.g. Microtrac S3500 Particle size analyser).

Preferably, the oxidation catalyst is supported with a support material as described and the supported catalyst comprises from 10 to 40% of the photoactive material by weight of the supported catalyst, preferably from 15 to 35%, more preferably 20 to 30% of the photoactive material by weight of the supported catalyst.

The loading of the thermally active component such as the previously mentioned metals in the oxidation catalyst is not particularly limited, and it will be appreciated that the amount of metals used may be limited by cost efficiency. Suitably, where the oxidation catalyst is supported on a support material, the one or more metals each may be present on the oxidation catalyst from 1% to 10% by weight of the catalyst, preferably from 2% to 5% by weight of the catalyst.

It will be appreciated that the supported catalyst may be used in the form of a packed bed of powder, granulates, pellets or extrudates over which the feed stream is passed.

The supported or unsupported oxidation catalyst may be loaded onto a physical support structure such as monolithic catalyst supports that are known to a person of skill in the art. It will be appreciated that a monolithic support as referred to herein is typically a structure comprising a plurality of channels through the structure, for example in a honeycomb structure. The channels may suitably be any shape, for example square, hexagonal or round. The density of channels may be any suitable range, and preferably from around 30 to 200 per cm². Preferably the wall thickness between channels is from 0.05 to 0.30 mm. Such an arrangement may provide a high open frontal area of from around 70 to 90% and can give rise to low backpressure in automotive exhaust systems.

The monolith may be made from any suitable material. The monolith may comprise a metal such as stainless steel, or in some embodiments may comprise a ceramic refractory material such as cordierite. It will be appreciated that the material of the monolith may in some instances be at least partially pervious to UV to allow better access of UV light to the coated internal channels of the monolith. For example, transparent ceramic materials are described in “M. Schulz, Adv. Appl. Ceram., 108, p 454 (2009)” and “X. Hao et al., Ceramics International, 41, p 14130 (2015)”.

The oxidation catalyst may be loaded onto the monolith in any suitable way. Suitably, the supported or unsupported oxidation catalyst is loaded onto the monolith as a washcoat. Such application of catalyst to a monolith as a washcoat is known to those of skill in the art, and may typically comprise dip coating the monolith in a slurry of the chemically supported or unsupported catalyst, followed by drying. The steps can be repeated to obtain the desired catalyst loading on the monolith, and the catalyst-loaded monolith may be subsequently calcined. An example of washcoating a monolith may be found, for example in “A. Scarabello et al., Applied Catalysis B: Environmental, 174-175, p 308 (2015)”. It will be appreciated that a supported catalyst will typically be in the form of a powder to facilitate application of the catalyst as a washcoat to a monolith.

In general, where a supported oxidation catalyst is loaded onto a monolith, the supported catalyst may be loaded so as to give about 15% to about 30% loading of the supported catalyst by weight of the catalyst and monolith. Preferably the oxidation catalyst loaded on a monolith comprises from 3 to 15% of the photoactive material by weight of the catalyst and monolith, preferably from 5 to 12%, more preferably 6 to 10% of the photoactive material by weight of the catalyst and monolith. Similarly, where a monolith is used, the oxidation catalyst loaded on the monolith may comprise from 0.3% to 4% by weight of the catalyst and monolith of a thermally active component, such as the one or more metals described previously, preferably from 0.5% to 2% by weight of the catalyst and monolith.

It will be appreciated that different components of the oxidation catalyst may be separately or simultaneously incorporated onto the same support material, or a thermally active component may be supported on the photoactive material to form the catalyst. For example, the one or more metals described previously may be impregnated on the photoactive material or, where the catalyst is supported, onto the support material. It will be appreciated that the steps of impregnation of the photoactive material and metals may be performed in any suitable sequence, and some steps may be conducted simultaneously. The metal may be added at one or more of the catalyst preparation stages including: during precipitation as a soluble compound; precipitation by incipient wetness impregnation; or following calcination of the catalyst. The photoactive material may be impregnated onto a support material or loaded onto a monolith at the same time as one or more metals, for example from the same impregnation solution or suspension or in the same washcoat step, or the metal may be impregnated onto a support in a separate step to the photoactive material. Alternatively, one or more metals may be impregnated onto the photoactive material, followed by impregnation of the photoactive material and metal onto a support material or loading onto a monolith.

In preferred embodiments, the oxidation catalyst further comprises one or more dispersion aids, strength aids and/or binders.

It will be appreciated that the source of the gaseous feedstream is not particularly limited and could be any suitable stream in which the oxidation of C1-C5 hydrocarbons is desired. In particular, the source of the gaseous feedstream may be any stream comprising methane, where it is desired to oxidise the methane. Preferably, the gaseous feedstream is an exhaust gas stream, for example an exhaust gas stream from a hydrocarbon combustion process, for example an exhaust stream from an internal combustion engine. It will be appreciated that the present method may be particularly advantageous when used with an engine that outputs an amount of methane in the exhaust gas, for example a dual fuel engine using diesel and a methane-based fuel such as natural gas, or a natural gas engine. Thus, it will be understood that the gaseous feedstream may suitably comprise an exhaust stream from a hydrocarbon combustion process using a methane-based fuel such as natural gas. A dual fuel engine may, for example, operate with a diesel intake of around 2.5 to 3 g per second, and a methane intake of around 1 to 3.5 g per second. Thus, the intake ratio of diesel to methane may suitably vary between about 5:1 and 1:2, for example between 3:1 and 1:1.5. Preferably, the exhaust gas stream is derived from: i) an engine, such as an engine powered by natural gas and/or propane; or ii) an electric power generator or combined heat and power (CHP) generator.

The term “natural gas” will be understood to refer to hydrocarbon fuels comprising a major proportion (i.e. above 50% by volume) of methane. Examples therefore include fuels comprising at least 60% by volume of methane, preferably at least 70% by volume of methane, more preferably at least 80% by volume of methane. Typically natural gas may comprise from 60 to 90% by volume of methane, with the balance comprising primarily C2-C5 hydrocarbons, nitrogen and carbon dioxide, and in some instances hydrogen sulfide.

In preferred embodiments, the engine is an internal combustion engine for an automotive vehicle, locomotive vehicle or marine vessel and steps a) and b) of the method are conducted adjacent to, or inside, the exhaust gas system of the automotive vehicle, locomotive vehicle or marine vessel.

“Automotive vehicle” will be understood to refer to any land-based vehicle, typically propelled at least in part by an internal combustion engine. For example, automotive vehicles may include cars, motorbikes, buses, trucks and lorries. The term “locomotive vehicle” will be understood to include any vehicle propelled along fixed tracks, for example trains or trams. The term “marine vessel” will be understood to include any water-based vehicle, for example boats, submarines, ships or tankers.

In a further aspect, the present invention provides an apparatus for use in catalytic oxidation of C1-C5 alkane present in a gaseous feedstream, said apparatus comprising:
a) an oxidation catalyst configured for catalytic oxidation of C1-C5 alkane present in a gaseous feedstream at temperatures of up to 600° C., wherein the catalyst comprises a photoactive material;
b) a UV light generating means configured for irradiation of the photoactive material; and
c) a housing within which the oxidation catalyst is disposed and within which UV light from the UV light generating means may be transmitted, which housing is configured to receive a supply of the gaseous feedstream comprising C1-C5 alkane.

It will be appreciated that the oxidation catalyst, the UV generating means/irradiation, the gaseous feedstream and any other elements of the apparatus may be substantially as defined previously herein.

The “housing” referred to herein will be understood to refer to any structural means for accommodating the oxidation catalyst and receiving/accommodating UV light from the UV light generating means and which is suitable for partial or temporary containment of the gaseous feedstream. The housing may correspond to a component of an exhaust system of an engine, or a part which is suitable for being retrofitted so as to form a component of an exhaust system of an engine.

The oxidation catalyst (supported or unsupported) is disposed within the housing, e.g. secured to an interior surface of the housing. As will be appreciated, in all configurations the housing will suitably have one or more inlets and one or more outlets through which the gaseous feedstream may enter and exit, respectively.

In some embodiments, the housing may form part of a support structure which is loaded with catalyst, for example a monolith where interior channels are loaded with the oxidation catalyst and which are capable of receiving UV light from a UV light generating means. As will be appreciated, in such an example, the presence of multiple inlets and outlets allowing the flow of gases may exist in the form of the interior channels of the monolith defining tortuous paths through which gases may flow. In alternative embodiments, the housing may be structurally distinct from, and enclose, an internal support structure, such as a monolith (e.g. where the housing corresponds to an external shell surrounding the support structure).

The oxidation catalyst is disposed within the housing so as to be contacted with the gaseous feedstream as it flows through the housing and the UV light generating means is configured to provide UV radiation to the catalyst during operation. Preferably, the oxidation catalyst is applied on a monolithic structure disposed within a housing. In other preferred embodiments, the UV light generating means may be external to the housing and may transmit UV light to the interior of the housing by means, for instance, of an optical fibre. In other embodiments, the means for generating the UV light is disposed inside the housing itself, for example, as a UV lamp.

It will be appreciated that the housing may be formed from a single section/compartment or a plurality of different sections/compartments. The housing may, for example, comprise a section/compartment within which the oxidation catalyst is disposed and a separate section/compartment within which the UV light generating means is disposed, provided the proximity of the different sections/compartments allows the UV light generating means to provide UV radiation to the oxidation catalyst contained within the housing.

Preferably, the gaseous feedstream is an exhaust gas and the housing is configured for fluidic attachment to a means for conveying the exhaust gas from an exhaust gas supply, such as piping connected to an engine's exhaust system. The housing and/or catalyst may be disposed at any suitable position in the exhaust train. Preferably, the housing within which the oxidation catalyst is disposed is upstream of a conventional catalytic converter, where present. A conventional catalytic converter may for instance comprise an SCR catalyst. Positioning the oxidation catalyst upstream of a conventional catalytic converter may reduce or avoid deactivation of the oxidation catalyst due to residual emissions from the catalytic converter, for example if excess ammonia from the SCR catalyst is present in the exhaust stream.

The engine may be substantially as defined previously herein. For example, the engine may power an automotive vehicle, locomotive vehicle or marine vessel and the apparatus is configured for integration adjacent to, or inside, the exhaust system of the automotive vehicle, locomotive vehicle or marine vessel.

The source of UV light may be arranged in any suitable way so as to irradiate the photoactive material of the oxidation catalyst. The irradiation may be direct irradiation or may be indirect, for example by reflection, refraction or diffraction of the UV light before it is incident on the catalyst. The UV light source may suitably be arranged upstream or downstream of the catalyst, or may be disposed between the upstream and downstream limits of the catalyst, for example the UV source may be disposed at least partially within the catalyst structure such as at least partially within a monolithic structure or packed bed. Where the UV light source is disposed within the catalyst structure, it may be particularly advantageous to provide the UV light source through one or more optical fibres, such that the bulk of the UV light generating means can be disposed elsewhere and the amount of displaced catalyst volume is reduced. In some embodiments, where a physically longer flow path through the catalyst is desired, for example where the feed stream flow is particularly high, the catalyst may be divided into segments, with a UV light source positioned between different catalyst segments, or UV light may be provided indirectly, for example through optical fibres, to multiple locations along the flow path through the catalyst.

In preferred embodiments, the UV light source comprises more than one separate source arranged to irradiate the photocatalyst. For example, the UV light source may comprise two or more UV sources offset from each other and arranged to provide overlapping irradiation at the centre of the catalyst structure. The UV light irradiated onto the photoactive material may be provided from an array of UV sources, for example an array of LED UV sources. It has been found by the inventors that most of the flow over the catalyst is through the centre of the catalyst structure. Thus, by providing overlapping irradiation as described, the activity of the catalyst at the centre of the catalyst structure, over which increased flow is present, may be improved.

In preferred embodiments, the housing comprises a UV light reflective interior surface. It will be appreciated that any suitable reflective surface may be used that can withstand the range of temperatures used in accordance with the invention. Preferably the reflective interior surface comprises metal foil, a metal sheet, a mirror, a lens such as a glass lens, fibre optics or ceramic. In some instances, a reflective surface may be provided to direct light from the UV light source towards the oxidation catalyst. For example where the UV light source emits light in multiple directions, some of which are not towards the oxidation catalyst, a reflective surface may be provided to direct a larger proportion of the UV light towards the oxidation catalyst. For example, where a UV light source is positioned upstream or downstream of the oxidation catalyst, a reflective surface may be used to direct the respective upstream or downstream light towards the catalyst. In some embodiments, one or more reflective surfaces may be used to direct the UV light from multiple UV light sources to a particular region of the oxidation catalyst where increased flow is present, to provide overlapping irradiation.

A further aspect provides an exhaust system for an internal combustion engine for powering an automotive vehicle, locomotive vehicle or marine vessel comprising an apparatus as defined previously herein.

The invention will now be further described by reference to the following Examples and with reference to the following figures, in which:

FIG. 1: shows a schematic of the lab-scale testing setup according to Examples 3 and 4;

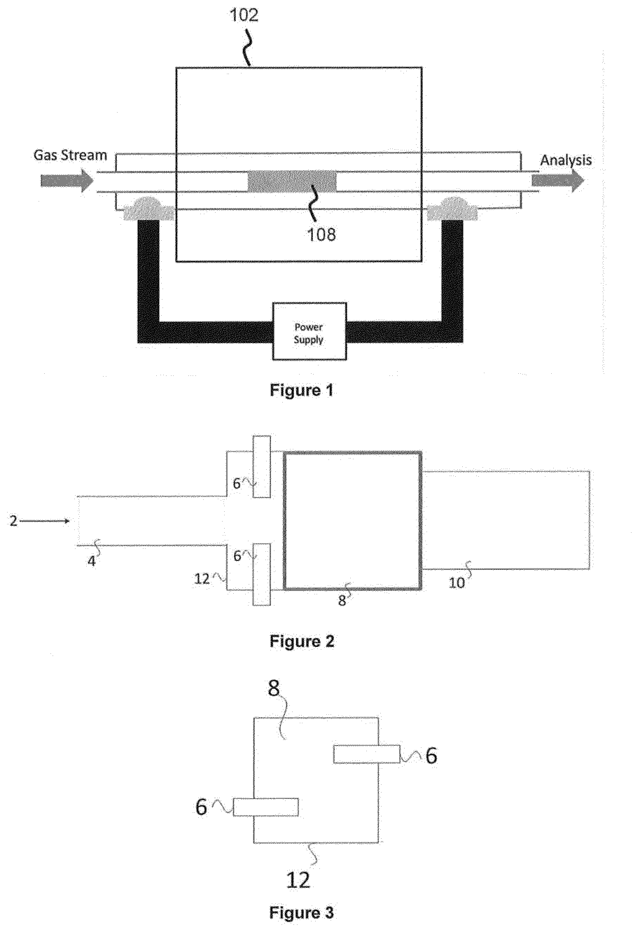

FIG. 2: shows a schematic of the testing setup with the catalyst integrated into a vehicle exhaust according to Example 5;

FIG. 3: shows a different schematic of the testing set-up with the catalyst integrated into a vehicle exhaust according to Example 5;

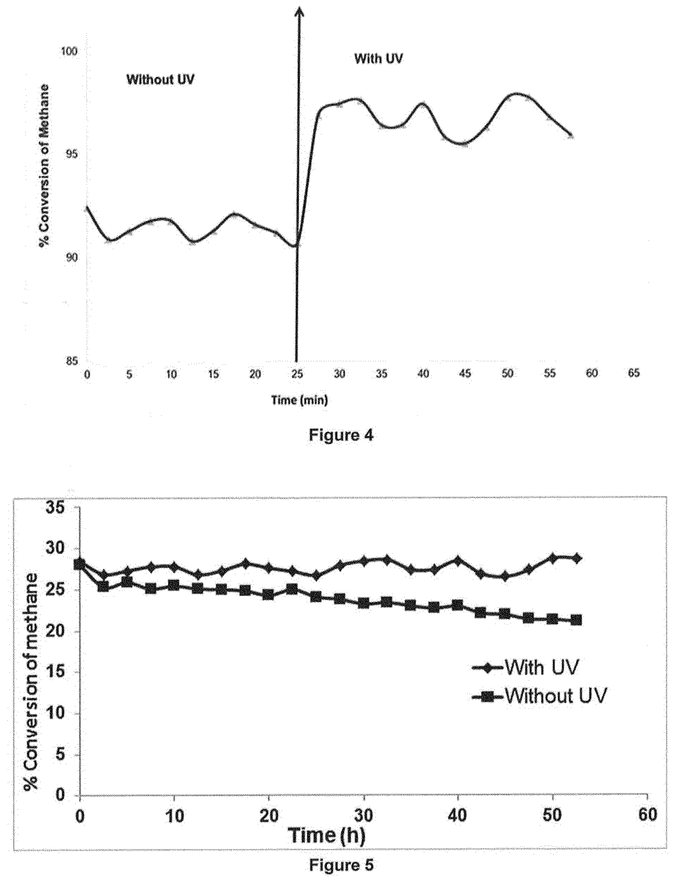

FIG. 4: is a graph showing the effect of UV irradiation in accordance with Example 6;

FIG. 5: is a graph showing the effect of UV irradiation in accordance with Example 7;

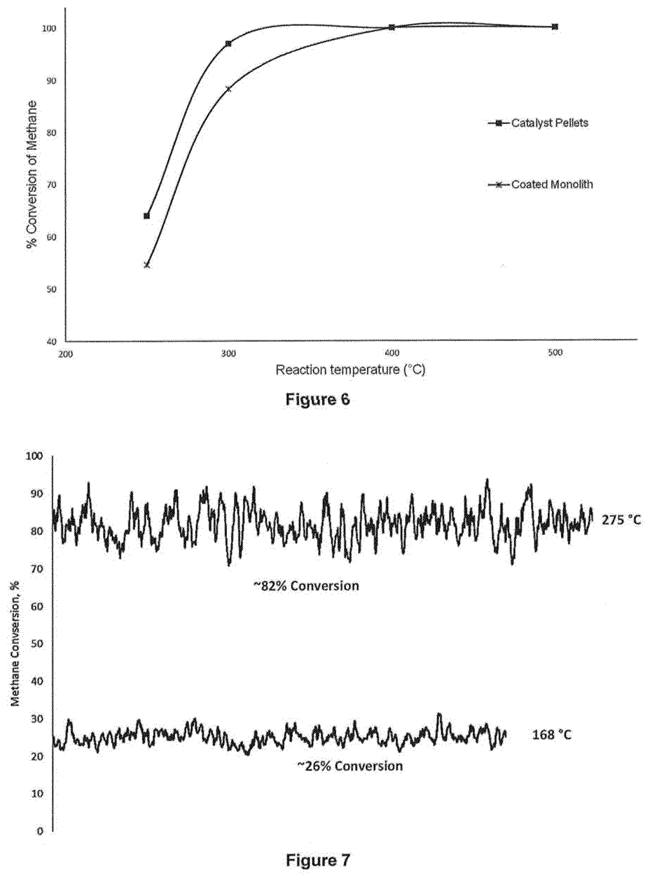

FIG. 6: is a graph showing a comparison of catalyst pellets with a coated monolith in accordance with Example 8;

FIG. 7: is a graph showing methane oxidation at different temperatures in accordance with Example 9;

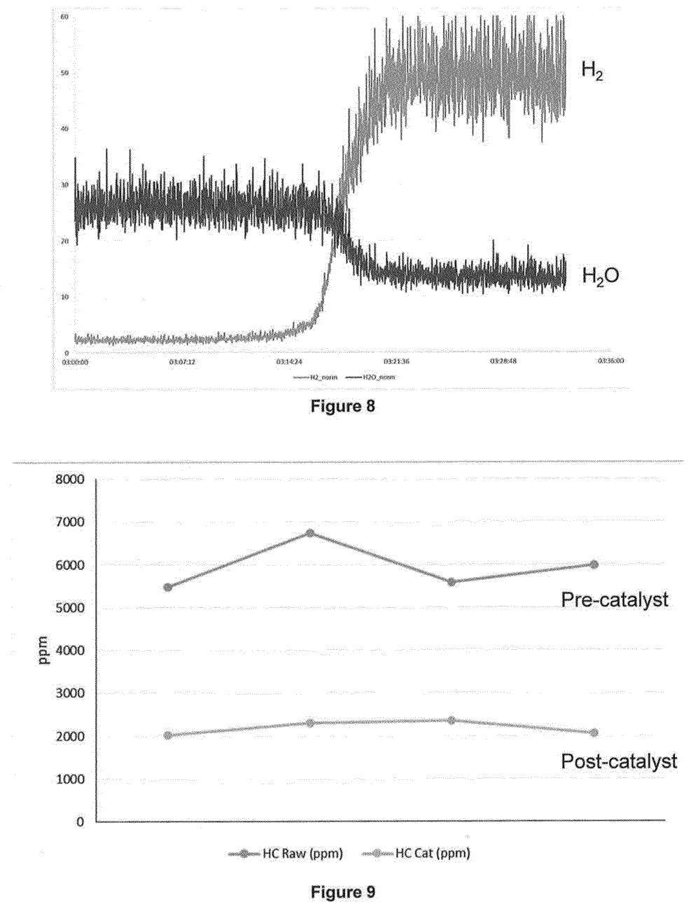

FIG. 8: is a graph showing conversion of water to hydrogen in accordance with Example 10;

FIG. 9: is a graph showing methane concentration in accordance with Example 11;

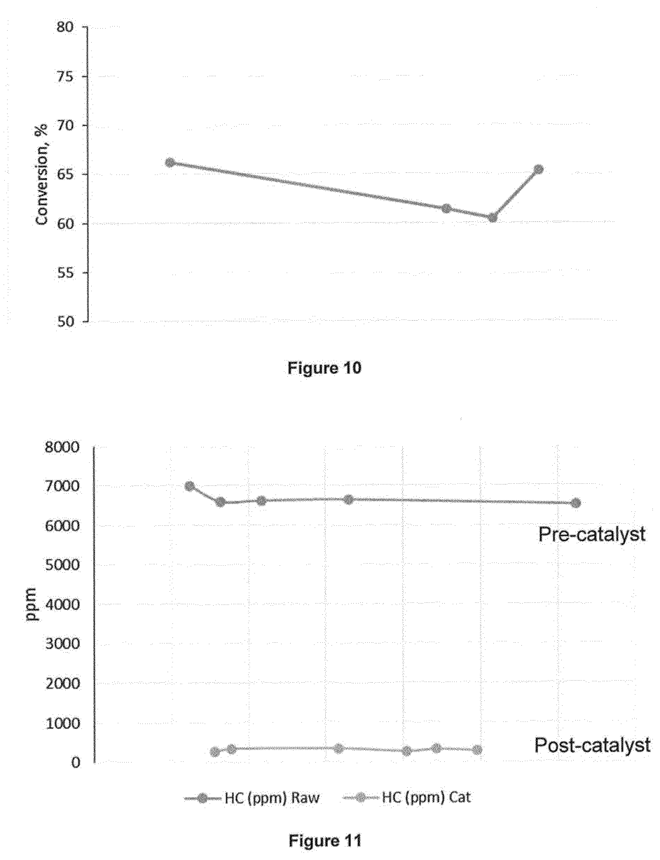

FIG. 10: is a graph showing methane conversion in accordance with Example 11;

FIG. 11: is a graph showing methane concentration in accordance with Example 12;

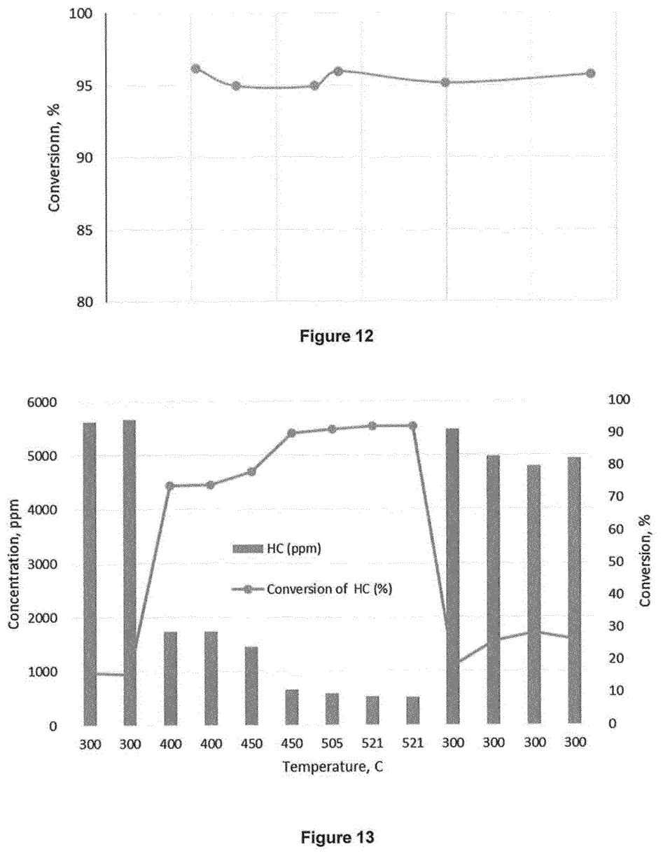

FIG. 12: is a graph showing methane conversion in accordance with Example 12; and

FIG. 13: is a graph showing methane conversion in accordance with Comparative Example 1.

### EXAMPLES

### Example 1

### Preparation of powdered catalyst

The catalyst was prepared by a wet impregnation method with the aid of sonication. ZSM-5 zeolite was placed in a vial and the mass of metal precursor (palladium nitrate dihydrate or tetraammineplatinum (II) hydroxide) solution or slurry, required to give a 5 wt. % palladium and 2 wt. % platinum loading was added to the powder. Then the required amount of TiO₂ to give 25 wt. % loading was added and the powder mixture dispersed in deionised water (5 ml). The mixture was sonicated at 80° C. (Crest ultrasonic bath model 200 HT), under a 45 kHz frequency for 3 h and resulted in a homogeneous paste. All mixtures were dried at 120° C. overnight in an oven before being calcined in air at 500° C. in a furnace for 4 h with a heating ramp of 2° C. min⁻¹. The powder catalyst had a particle size in the range of 250 to 425 μm.

### Example 2

### Preparation of Catalyst Monolith

The catalyst prepared according to Example 1 was loaded onto a cordierite monolith (400 cpsi/62 cells per cm², wall thickness 0.07 mm) as a washcoat, so as to give a catalyst loading of 18.6 wt. % on the monolith.

### Example 3

### General Procedure for Lab-Scale Tests

FIG. 1 shows a schematic representation of the lab-scale setup for catalyst testing. With reference to FIG. 1, a gaseous feedstream is supplied into a quartz sample tube (22 mm outer diameter) containing a packed bed of powdered catalyst 108 prepared according to the general procedure above or a coated monolith. The quartz sample tube containing the catalyst is disposed inside a stainless steel tube which in itself is disposed inside a tubular furnace 102 for heating the catalyst and feedstream, and UV light sources are arranged on both sides of the catalyst, inside the steel tube but outside the quartz tube, for irradiating the catalyst. The gaseous stream exiting the tube after passing over the catalyst is then sent to a mass spectrometer for analysis. Where a monolith is used, the packed bed of powdered catalyst is replaced with a monolith cut to fit the stainless steel tube with a monolith length of 20 mm.

The gaseous feedstream was 0.5% methane, 10% oxygen, 5% Neon, dry or 1 to 10% water, and the remaining balance of argon. Methane conversion was calculated by comparing against a baseline with no catalyst and a sample of the feed taken upstream of the catalyst.

### Example 4

### General Prcedure for Chassis Dyno Tests

The setup in FIG. 1 and described in Example 3, was used to analyse a feedstream sampled from the exhaust of an engine, just after the turbo. The engine was a DAF Truck 9 L, diesel/natural gas dual fuel engine, operated in dual fuel mode with an approximately 50:50 blend of diesel and natural gas.

### Example 5

### General Procedure/Setup for Monolith Testing Integrated in an Exhaust

FIG. 2 shows a schematic representation of the setup for testing where the photocatalyst is integrated into the exhaust train of a HGV (DAF truck with 9 L, diesel/natural gas dual fuel engine, operated in dual fuel mode with an approximately 50:50 blend of diesel and natural gas). An exhaust stream 2 is directed down a pipe 4 from the turbo of the engine into housing 12. UV lights 6 are disposed adjacent a catalyst monolith 8 prepared according to Example 2. The exhaust gases 2 pass through the catalyst monolith 8 and are passed to a conventional catalytic convertor 10. Exhaust gases were analysed using an exhaust gas analyser for real-time concentration measurements (Kane International Limited 4 gas analyser and mass spectrometry).

As shown in FIG. 3, the UV lights 6 are generally offset from the perimeter of the monolith inlet and are offset from each other so as to provide overlapping UV irradiation to the central area of the monolith.

### Example 6

### Effect of UV Light on Catalyst Activity

An experiment was conducted using the setup of Example 3, with a dry feedstream at a GHSV of 100,000 mLg⁻¹h⁻¹ and using the catalyst of Example 1. The conversion of methane, as measured by mass spectroscopy, is shown over time in FIG. 4.

After around 25 minutes, the catalyst was illuminated continuously with UV light. Following this, an improvement in methane conversion of around 5% was observed, demonstrating an increase in catalyst activity when UV irradiation of the catalyst is used.

### Example 7

### Effect of UV Light on Catalyst Stability

Two experiments were conducted using the setup of Example 3 using a feed containing water vapour (1 to 10%) and the powdered catalyst according to Example 1: (i) with continuous irradiation of the catalyst with UV light and (ii) without UV irradiation of the catalyst. As can be seen in FIG. 5, with UV irradiation of the catalyst, the catalyst performance does not substantially decrease on a timescale of over 50 hours. Meanwhile, when the same catalyst is not irradiated with UV light, the catalyst performance degrades over time to give around an 8% decrease in methane conversion after about 50 hours compared to where UV irradiation is used.

### Example 8

### Lab-Scale Comparison of Catalyst Pellets and Monolith

Two experiments were conducted using the setup of Example 3 and a dry feed, one using a packed bed of powder and the other using a coated monolith. UV irradiation of the catalyst was conducted continuously. The conversion of methane at different temperatures is shown in FIG. 6. As can be seen, while the catalyst powder appears to achieve slightly better conversion at lower temperatures, the performance of the catalyst in the case of both pellets in a packed bed and with the coated monolith are comparable.

### Example 9

### Effect of Temperature on Methane Conversion

An experiment was conducted by sampling exhaust gases according to Example 4, using the powdered catalyst according to Example 1 and continuous UV irradiation of the catalyst. The reaction temperature was maintained at around 275° C. for approximately 1 hour, after which the temperature was lowered to 168° C. over a period of about 15 minutes. The conversion of methane over time at the two temperatures is shown in FIG. 7. At 275° C. the conversion corresponds to around 82% methane conversion, and even as low as 168° C. a methane conversion of around 26% is maintained.

### Example 10

### Water Conversion to Hydrogen

An experiment was conducted according to Example 3, using a freshly prepared catalyst according to Example 1 and at a feed temperature of 400° C. The catalyst was irradiated with UV light, and hydrogen and water content after the catalyst were measured by mass spectroscopy. A slight exotherm from 400° C. to about 412° C. was observed along with conversion of water into hydrogen, as illustrated by the change in water and hydrogen content as measured by mass spectroscopy, and shown in FIG. 8. When this experiment is conducted without irradiation with UV light, hydrogen production is not observed.

### Example 11

### Testing in Exhaust of a HGV

An experiment was conducted according to Example 5 and the methane concentration was monitored before (raw emissions) and after the catalyst. Continuous UV irradiation of the catalyst was used. Methane concentration before and after the catalyst for an exhaust stream at 260° C. produced by running the engine with a lambda value of 3 over a period of time of 2 to 3 hours, is shown in FIG. 9 and the corresponding % conversion of methane is shown in FIG. 10. As can be seen, under these conditions, methane conversions of from around 60% to 70% can be achieved.

### Example 12

### Testing in Exhaust of a HGV

An experiment was conducted according to Example 5 and the methane concentration was monitored before (raw emissions) and after the catalyst. Continuous UV irradiation of the catalyst was used. Methane concentration before and after the catalyst for an exhaust stream at 325° C. produced by running the engine with a lambda value of 4 over a period of time of 2 to 3 hours, is shown in FIG. 11 and the corresponding % conversion of methane is shown in FIG. 12. As can be seen, under these conditions, methane conversions of more than 95% can be achieved.

### Comparative Example 1

An experiment was conducted according to Example 5 and the methane concentration was monitored before (raw emissions) and after the catalyst. In this example no UV irradiation was used. For temperatures higher than 400° C., the engine was run with a lambda value of 1.5. The results are shown in FIG. 13. As can be seen, at 300° C. the conversion is significantly lower than at temperatures higher than 400° C., indicating that without UV irradiation, the water containing exhaust stream requires higher temperatures to reach the same conversion levels.

1. A method for the catalytic oxidation of a C1-C5 alkane using an oxidation catalyst comprising a photoactive material, said method comprising the steps of:
a) activating the photoactive material by irradiating the catalyst with UV light; and
b) contacting the activated catalyst with a gaseous feedstream comprising an amount of C1-C5 alkane at a temperature of from 150° C. to 600° C.

2. A method according to claim 1, wherein the photoactive material is activated in step a) in the absence of the gaseous feedstream comprising an amount of C1-C5 alkane, for example wherein activation is performed in the presence of air or under at least a partial vacuum.

3. A method according to claim 1, wherein the photoactive material is activated in step a) in the presence of the gaseous feedstream comprising an amount of C1-C5 alkane, and preferably at a temperature of from 150° C. to 600° C.

4. A method according to any one of claims 1 to 3, wherein the photoactive material is irradiated intermittently or continuously with UV light during contact with the gaseous feedstream comprising an amount of C1-C5 alkane in step b).

5. A method according to any one of the preceding claims, wherein the gaseous feedstream comprises from 0.01 to 20% by volume of C1-C5 alkane, preferably from 0.1 to 10.0% by volume, more preferably from 0.5 to 5.0% by volume of C1-C5 alkane.

6. A method according to any one of the preceding claims, wherein the C1-C5 alkane is selected from C1-C3 alkanes and combinations thereof, more preferably the C1-C5 alkane is selected from methane, propane or a combination thereof, even more preferably the C1-C5 alkane is methane.

7. A method according to any one of the preceding claims, wherein the feedstream comprises from 4.0% to 20.0% by volume of water vapour, preferably from 5.0 to 15.0% by volume of water vapour, more preferably from 5.0% to 10.0% by volume of water vapour.

8. A method according to any one of the preceding claims, wherein contacting step b) is conducted at a temperature of at least 175° C., preferably at least 200° C., more preferably at least 225° C., even more preferably at least 250° C.

9. A method according to any one of the preceding claims, wherein irradiation in step a) is with UV radiation having a wavelength of from 280 to 450 nm, preferably UV radiation having a wavelength of from 375 to 395 nm.

10. A method according to any one of the preceding claims, wherein the photoactive material comprises a photoactive material selected from TiO₂, WO₃ and CoO and combinations thereof, preferably wherein the photoactive material is TiO₂.

11. A method according to any one of the preceding claims, wherein the oxidation catalyst comprises one or more metals selected from ruthenium, palladium, platinum, gold, silver, rhodium, iridium, rhenium, manganese, chromium, nickel, iron, molybdenum, tungsten, zirconium, gallium, thorium, lanthanum, cerium and mixtures thereof.

12. A method according to claim 11, wherein the oxidation catalyst comprises at least two different metals, preferably selected from palladium, platinum, gold, silver, rhodium, iridium and rhenium, more preferably wherein the at least two different dopant metals includes palladium and platinum.

13. A method according to any one of the preceding claims, wherein the oxidation catalyst is supported with a support material, wherein the support material is in the form of a powder, granulate, pellet, extrudate, or combinations thereof.

14. A method according to claim 13, wherein the catalyst is supported with a support material and the supported catalyst comprises from 10 to 40% of the photoactive material by weight of the supported photocatalyst, preferably from 15 to 35%, more preferably 20 to 30% of the photoactive material by weight of the supported catalyst.

15. A method according to claim 13 or claim 14, wherein the support material is selected from silica, alumina, aluminosilicate such as zeolite, silica-alumina, ceria, titania, gallia, zirconia, magnesia, zinc oxide, activated carbon, silicon carbide, titanium carbide, fluoropolymer resins and mixtures thereof, preferably where the support material is alumina or zeolite such as ZSM-5.

16. A method according to any one of the preceding claims wherein the gaseous feedstream is an exhaust gas stream, preferably wherein the exhaust gas stream is derived from: i) an engine, such as an engine powered by natural gas and/or propane; or ii) an electric power generator or combined heat and power (CHP) generator.

17. A method according to claim 16, wherein the engine is an internal combustion engine for an automotive vehicle, locomotive vehicle or marine vessel and steps a) and b) of the method are conducted adjacent to, or inside, the exhaust gas system of the automotive vehicle, locomotive vehicle or marine vessel.

18. A method according to any one of claims 1 to 15, wherein the gaseous feedstream is an exhaust stream from a hydrocarbon combustion process using a methane-based fuel.

19. An apparatus for use in catalytic oxidation of C1-C5 alkane present in an gaseous feedstream, said apparatus comprising:
a) an oxidation catalyst configured for catalytic oxidation of C1-C5 alkane present in a gaseous feedstream at temperatures of up to 600° C., wherein the catalyst comprises a photoactive material;
b) a UV light generating means configured for irradiation of the photoactive material; and
c) a housing within which the oxidation catalyst is disposed and within which UV light from the UV light generating means may be transmitted, which housing is configured to receive a supply of the gaseous feedstream comprising C1-C5 alkane.

20. An apparatus according to claim 19, wherein the gaseous feedstream is an exhaust gas and the housing is configured for fluidic attachment to a means for conveying the exhaust gas from an exhaust gas supply, such as piping connected to an engine's exhaust system.

21. An apparatus according to claim 19 or claim 20, wherein the engine powers an automotive vehicle, locomotive vehicle or marine vessel and the apparatus is configured for integration adjacent to, or inside, the exhaust system of the automotive vehicle, locomotive vehicle or marine vessel.

22. An apparatus according to any one of claims 19 to 21, wherein the housing comprises a UV light reflective interior surface, such as a metal foil or ceramic.

23. An apparatus according to any one of claims 19 to 22, wherein the oxidation catalyst is as defined in any one of claims 10 to 15.

24. An apparatus according to claim 23, wherein the oxidation catalyst is applied on a monolithic structure disposed within the housing.

25. An apparatus according to any one of claims 19 to 24, wherein the UV light generating means is configured to provide pulsed and/or continuous UV light having a wavelength of from 280 to 450 nm, preferably having a wavelength of from 375 to 395 nm.

26. An exhaust system for an internal combustion engine for powering an automotive vehicle, locomotive vehicle or marine vessel comprising an apparatus as defined in any one of claims 19 to 25.
